SEQUENCE LISTING (11 sequences)

SEQ ID NO: 1 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 2 - protein, 22 aa, Homo sapiens
KRSNYTPITNVPPEVTVLTNSP

SEQ ID NO: 3 - protein, 25 aa, Homo sapiens
KFTPPVVNVTWLRNGKPVTTGVSET

SEQ ID NO: 4 - protein, 22 aa, Homo sapiens
LLKHWEFDAPSPLPETTENVVC

SEQ ID NO: 5 - protein, 614 aa, Homo sapiens
MTFLISYWGEQIGQKIRKITDCFHCHVFPFLQQEEARLGALQQLQQQSQELQEVLGETERFLSQVLGRVLQLLPPGQVQVHKMKAVYLALNQCSVSTTHKCLIAEAWCSVRDLPALQEALQDSSMEEGVSAVAHRIPCRDMPPTLIRTNRFTASFQGIVDAYGVGRYQEVNPAPYTIITFPFLFAVMFGDVGHGLLMFLFALAMVLAENRPAVKAAQNEIWQTFFRGRYLLLLMGLFSIYTGFIYNECFSRATSIFPSGWSVAAMANQSGWSDAFLAQHTMLTLDPNVTGVFLGPYPFGIDPIWSLAANHLSFLNSFKMKMSVILGVVHMAFGVVLGVFNHVHFGQRHRLLLETLPELTFLLGLFGYLVFLVIYKWLCVWAARAASAPSILIHFINMFLFSHSPSNRLLYPRQEVVQATLVVLALAMVPILLLGTPLHLLHRHRRRLRRRPADRQEENKAGLLDLPDASVNGWSSDEEKAGGLDDEEEAELVPSEVLMHQAIHTIEFCLGCVSNTASYLRLWALSLAHAQLSEVLWAMVMRIGLGLGREVGVAAVVLVPIFAAFAVMTVAILLVMEGLSAFLHALRLHWVEFQNKFYSGTGYKLSPFTFAATDD

SEQ ID NO: 6 - protein, 13 aa, Homo sapiens
RRPADRQEENKAG

SEQ ID NO: 7 - protein, 29 aa, Homo sapiens
RRPADRQEENKAGLLDLPDASVNGWSSDE

SEQ ID NO: 8 - protein, 14 aa, Homo sapiens
DLPDASVNGWSSDE

SEQ ID NO: 9 - protein, 24 aa, Homo sapiens
DLPDASVNGWSSDEEKAGGLDDEE

SEQ ID NO: 10 - protein, 24 aa, Homo sapiens
VEFQNKFYSGTGYKLSPFDFAATD

SEQ ID NO: 11 - protein, 14 aa, Homo sapiens
VEFQNKFYSGTGYK

(19) United States
(12) Patent Application Publication
(10) Pub. No.: US 2005/0048067 A1
(43) Pub. Date: Mar. 3, 2005

(54) Peptides capable of modulating immune response

(76) Inventor: Nalan Utku, Berlin (DE)

Correspondence Address: Cooper & Dunner, 1185 Avenue of the Americas, New York, NY, 10036, US

(21) Appl. No.: 10/489,725

(22) PCT Filed: Sep. 17, 2002
(86) PCT No.: PCT/EP02/10440

Related U.S. Application Data
(60) Provisional application No. 60/322,896, filed on Sep. 17, 2001.
(60) Provisional application No. 60/322,895, filed on Sep. 17, 2001.

(51) Int. Cl. A61K 39/00; C07H 21/04; C07K 14/74

(52) U.S. Cl. 424/185.1; 435/69.1; 435/320.1; 435/325; 530/350; 536/23.5

(57) ABSTRACT

Provided are peptides capable of inhibiting proliferation of peripheral blood mononuclear cells (PBMCs) derived from HLA class II alpha 2 chain. Furthermore, peptides capable of inhibiting proliferation of peripheral blood mononuclear cells (PBMCs) derived from T-cell immune response cDNA7 (TIRC7) costimulatory molecule are described. Compositions comprising such peptides and their use for the treatment of immune diseases are provided.

The present invention relates to peptides capable of modulating the function (e.g., signalling or adhesive activities) of T-cell immune response cDNA 7 (TIRC7) and/or its ligand. In particular, the peptides of the invention are capable of suppressing the proliferation of activated cells of the immune system. Furthermore, the present invention relates to compositions comprising said peptides and to methods of modulating immune cell proliferation, and treating immune response related diseases.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

T-cell activation is a serial process involving multiple signaling pathways and sequential changes in gene expression resulting in differentiation of T-cells into distinct subpopulations, i.e. Th1 and Th2, which are distinguishable by their pattern of cytokine production and characterize the mode of cellular immune response. The T-cell response is initiated by the interaction of the antigen-specific T-cell receptor (TCR) with peptide presented by major histocompatibility complex (MHC) molecules on the surface of antigen presenting cells (APCs). Additional signals are provided by a network of receptor-ligand interactions mediated by a number of membrane proteins such as CD28/CTLA4 and B7, CD40/CD40L, LFA-1 and ICAM-1 (Lenschow, Science 257 (1992), 789-792; Linsley, Annu. Rev. Immunol. 11 (1993), 191-212; Xu, Immunity 1 (1994), 423-431; Bachmann, Immunity 7 (1997), 549-557; Schwartz, Cell 71 (1992), 1065-1068) collectively called costimulatory signals (Perez, Immunity 6 (1997), 411). These membrane proteins can alter T-cell activation in distinct ways (Bachmann, Immunity 7 (1997), 549-557) and regulate the immune response by the integration of positive and negative signals provided by these molecules (Bluestone, Immunity 2 (1995), 555-559; Perez, Immunity 6 (1997), 411). Many of the agents which are effective in modulating the cellular immune response either interfere with the T-cell receptor (Cosimi, Transplantation 32 (1981), 535-539) block costimulatory signaling (Larsen, Nature 381 (1996), 434-438; Blazar J. Immuno. 157 (1996), 3250-3259; Kirk, Proc. Natl. Acad. Sci. USA 94 (1997), 8789-8794; Linsley, Science 257 (1992), 792-95; Turka, Proc. Natl. Acad. Sci. USA 89 (1992), 11102-11105) or inhibit intracellular activation signals downstream from these primary cell membrane triggers (Schreiber and Crabtree, Immunology Today 13 (1992), 136-42). Therapeutic prevention of T-cell activation in organ transplantation and autoimmune diseases presently relies on panimmunosupressive drugs interfering with downstream intracellular events. Specific modulation of the immune response remains a longstanding goal in immunological research.

In view of the need of therapeutic means for the treatment of diseases related to immune responses of the human body, the technical problem of the present invention is to provide means and methods for modulation of the immune response in a subject. The solution to said technical problem is achieved by providing the embodiments characterized in the claims, and described further below.

Accordingly, in a first aspect the present invention relates to a peptide capable of inhibiting proliferation of peripheral blood mononuclear cells (PBMCs), comprising a fragment of the amino acid sequence from HLA-(Human Leukocyte associated Antigen) class II alpha 2 chain.

In a further aspect, the present invention relates to a peptide capable of inhibiting proliferation of peripheral blood mononuclear cells (PBMCs), comprising a fragment of the amino acid sequence from T cell immune response cDNA 7 (TIRC7) protein.

HLA class II is a heterodimer of two transmembrane glycoproteins, the alpha and beta chains. Oriented with their amino terminal ends on the outside of the cell, both chains comprise two extracellular domains, each of 90-100 amino acids, connected to a short cytoplasmic tail by a hydrophobic acid sequence that makes a single pass through the cell membrane. In the alpha chain the membrane distal domain is known as alpha 1 and the membrane proximal domain as alpha 2. Likewise, in the beta chain the membrane distal domain is known as beta 1 and the membrane proximal domain as beta 2. Both membrane proximal domains possess structural characteristics of C1-type immune globulin domains. The alpha 1 and beta 1 domains are polymorph and occupied with presentations of peptides (12-24mers) to the T-cell receptor during the course of T-cell activation. Studies using site-specific mutants have mapped the site of CD4 binding to the membrane proximal beta 2 domain of the HLA class II molecule.

Expression of certain HLA class II molecules and their polymorphism are strongly associated with a number of diseases such as insulin-dependent diabetes mellitus, Goodpasture syndrome, Pemphigus vulgaris, Systemic lupus erythramatosus, Multiple sclerosis, Grave's disease, Rheumatoid arthritis and Myastenia gravis.

Many of the disease associated with HLA polymorphismus do not involve active infections and their symptoms are caused by a chronic state of inflammation and/or autoimmunity. In the case of diabetes, one general mechanism which has been proposed is that T-cell is activated by presentation of a microbial antigen subsequently crossreact with self peptides to which they were tolerant before activation. Thus, Coxackie virus infections have been correlated with diabetes and various bacterial infections of the intestine with the HLA class II associated diseases.

In infectious diseases relatively little is known of the effects of HLA polymorphism. From studies on cohorts of AIDS patients HLA effect can be seen, such as quicker progression of the disease (Roger et al., Faseb, 12, 1998, p. 625-32, Marsh, Parham and Barber, “The HLA-FACS-Book”, Academic Press (2000), 37-97). During the development the human response becomes tolerant of the normal components of healthy cells and tissues to which lymphocytes are exposed. Of the particular importance is that a person's immune system develops tolerance to the self HLA class I and II allotypes expressed on the surface of that same person's cell. By contrast a person's immune system is not tolerant of the many hundreds of non-self HLA allotypes expressed by other human beings such as after organ transplantation. Therefore, once a person receives a transplant, hyperacute or acute rejection of the transplanted organ is likely to occur if the recipient and donor are not compatible in their HLA antigen types expressed on the cell surface.

Without intending to be bound by theory, it is believed that the peptides derived from HLA class II alpha 2 chain or TIRC7 and described herein are capable of modulating the function (e.g., signaling or adhesive activities) of TIRC7, its family members and/or their ligands, for example by interfering with the interaction of TIRC7 with its ligand.

The term “interfering with the interaction of TIRC7 with its ligand” means in accordance with the present invention that said peptide or analog or derivative thereof is capable of inhibiting and/or modulating the interaction of TIRC7 with its corresponding ligand. Since the interaction of TIRC7 with its ligand(s) modulates events which are valuable in course of immune responses such peptide should also be capable of modulating immune responses. In accordance with the present invention said peptide preferably interacts with the TIRC7-ligand, for example by specifically binding to said ligand. The term “ligand” includes small molecules and soluble binding proteins as well a membrane associated receptors. “Specifically binding” means “specifically interacting with” whereby said interaction may be, inter alia, covalently, non-covalently and/or hydrophobic. However, irrespective the theory behind the molecular mechanism of action, the peptides of the invention can be characterized by (1) having at least 6, preferably 10, more preferably 12, still more preferably 15 or 20, and most preferably 25 to 30 consecutive amino acids of the amino acid sequence of the HLA class II alpha 2 chain or of TIRC7, and (2) being capable of inhibiting proliferation of mitogen-stimulated PBMCs in an assay as described in Example 1 and 3.

The term “TIRC7” as used in accordance with the present invention, denotes a protein involved in the signal transduction of T-cell activation and/or proliferation and that, preferably in a soluble form is capable of inhibiting or suppressing T-cell proliferation in response to alloactivation in a mixed lymphocyte culture or in response to mitogens when exogeneously added to the culture. In vitro translated TIRC7 protein is able to efficiently suppress in a dose dependent manner the proliferation of T-cells in response to alloactivation in a mixed lymphocyte culture or in response to mitogens. TIRC7 is known to the person skilled in the art and described, inter alia, in WO99/11782 and in Utku et al., Immunity 9 (1998), 509-518.

In one embodiment the HLA class II peptide of the present invention comprises a fragment of the amino acid sequence shown in FIG. 1. Active peptides could be larger or smaller than the ones specifically described here. While the present peptides described are of about 20 amino acids, peptides containing a relatively large number of amino acid residues, e.g., up to about 50 or 100 amino acid residues or more, that contain the described peptides, portions thereof, or similar peptides may have biological activity as well. Similarly, peptides smaller than those shown in FIG. 1 may also have similar biological activity. Similarly, peptides with amino acid substitutions or other alterations may block the activities of the described peptides or the parent molecules. Cyclic or otherwise modified forms of the peptides would also be expected to have biological activity. Preferably, the peptides of the present invention have about 12 to 30 amino acids derived from HLA class II alpha chain 2.

Thus, the present invention provides isolated peptides that include an amino acid sequence represented in FIG. 1 or analogs thereof that modulate the function of at least one TIRC7 protein and/or at least one ligand thereof. These amino acid sequences can form a part of a larger peptide. Additionally, they can be used in various combinations in any one peptide. Preferably, the present invention provides isolated peptides indicated as alpha 2 (a) or alpha 2 (b) in FIG. 1 or analogs thereof. It is believed that these portions of certain of the peptides described herein contribute significantly to the activity of the peptides.

In another embodiment the peptide of the present invention is derived from a TIRC7 protein which comprises an amino acid sequence as shown in FIG. 4(A), or a fragment thereof. Active peptides could be larger or smaller than the ones specifically described here. While the present peptides described are of about 13 to 29 amino acids, peptides containing a relatively large number of amino acid residues, e.g., up to about 50 or 100 amino acid residues or more, that contain the described peptides, portions thereof, or similar peptides may have biological activity as well. Similarly, peptides smaller than those shown in SEQ ID NOs: 6 to 10 may also have similar biological activity. Similarly, peptides with amino acid substitutions or other alterations may block the activities of the described peptides or the parent molecules. Cyclic or otherwise modified forms of the peptides would also be expected to have biological activity. Preferably, the peptides of the present invention have about 12 to 30 amino acids derived from TIRC7.

In addition, the present invention provides isolated peptides that include an amino acid sequence represented by SEQ ID NOs: 6 to 10 or analogs thereof that modulate the function of at least one TIRC7 protein and/or at least one ligand thereof. These amino acid sequences can also form a part of a larger peptide. Additionally, they can be used in various combinations in any one peptide, any one of HLA class II peptides. Preferably, the present invention provides isolated peptides represented by SEQ ID NOs: 6 to 10 or analogs thereof. It is believed that these portions of certain of the peptides described herein contribute significantly to the activity of the peptides.

The present peptides are preferably capable of modulating, preferably inhibiting proliferation of peripheral blood mononuclear cells (PBMCs). Preferably, the peptides of the present invention modulate at least one of the following (which are functions of TIRC7 proteins and/or ligands thereof): activation of neutrophils; activation or inhibition of T-cells, B-cells, NK cells, LAK cells, dendritic cells, or other immune system cells; proliferation and/or differentiation of T-cells, B-cells, NK cells, LAK cells, dendritic cells, or other immune system cells; proliferation and/or differentiation of epithelial cells such as breast or intestinal/colonic epithelium cells or keratinocytes. In addition these peptides are preferably capable of altering homotypic and/or heterotypic adhesion among TIRC7 proteins (i.e., TIRC7 family members) or adhesion of TIRC7 proteins to other TIRC7 ligands.

The present invention also provides peptide conjugates. The ability of peptides complexed with carrier molecules or structures, such as microbeads, liposomes, biological carrier molecules, synthetic polymers, biomaterials, and cells, thereby forming peptide conjugates is shown to impart the larger structure with the ability to bind to cells expressing TIRC7 or its ligand. Such peptide conjugates bind to cells expressing a TIRC7 protein or a TIRC7 ligand.

The peptides or peptide conjugates of the present invention can also include molecules for labeling (i.e., labels such as fluroescence tags, magnetic resonance tags, radioactive tags, enzymatic tags). In this way, these can be used in diagnostic methods to detect specific targets in vivo or in vitro.

The present invention also provides a method of modulating (e.g., activating or inhibiting) immune cell (e.g., T-cells, B-cells, NK cells, LAK cells, or dendritic cells) activation, proliferation, and/or differentiation that includes contacting an immune cell with a peptide or peptide conjugate described above. Preferably, the peptide is one of those indicated as alpha 2 (a) or alpha 2 (b) in FIG. 1 or a peptide as represented by SEQ ID NOs: 6, 7, 8, 9, or 10.

In addition, some peptides differ from these peptides by one or several amino acids and could compete with these active peptides or the natural TIRC7 protein or ligand thereof for certain biological activities.

Another method involves delivering a therapeutically active agent to a patient. The method includes administering at least one peptide or peptide conjugate comprising a peptide and the therapeutically active agent to a patient wherein the peptide preferably includes an amino acid sequence of the alpha 2 (a) or alpha 2(b) peptide or includes an amino acid sequence represented by SEQ ID Nos: 6 to 10 or analogs thereof. Preferably, the therapeutically active agent is selected from drugs, DNA sequences, RNA sequences, proteins, lipids, and combinations thereof. More preferably, the therapeutically active agent is an antibacterial agent, antiinflammatory agent, or antineoplastic agent.

Yet another method of the present invention involves altering an immune response. The method includes contacting immune system cells with at least one peptide or peptide conjugate of the present invention described above.

The methods described herein can be carried out in vitro or in vivo. The peptides can be used alone or in various combinations as well as in peptide conjugates. They are used in amounts that provide the desired effect. These amounts can be readily determined by one of skill in the art. Preferably, for each of the methods of the present invention, useful peptides are represented by SEQ ID NOs: 3, 4, 6, 7, 8, 9 or 10.

As used herein, “a” or “an” refers to one or more of the term modified. Thus, the compositions and methods of the present invention include one or more peptides. Also, herein when peptide is said to includes an amino acid sequence preferably includes an amino acid sequence of the alpha 2 (a) or alpha 2(b) peptide or as represented by SEQ ID Nos: 6 to 10 or analogs thereof, the peptide can include one or more of the sequences specified. “Amino acid” is used herein to refer to a chemical compound with the general formula: NH₂—CRH—COOH, where R, the side chain, is H or an organic group. Where R is an organic group, R can vary and is either polar or nonpolar (i.e., hydrophobic). The amino acids of this invention can be naturally occurring or synthetic (often referred to as nonproteinogenic). As used herein, an organic group is a hydrocarbon group that is classified as an aliphatic group, a cyclic group or combination of aliphatic and cyclic groups.

The term “aliphatic group” means a saturated or unsaturated linear or branched hydrocarbon group. This term is used to encompass alkyl, alkenyl, and alkynyl groups, for example.

The term “cyclic group” means a closed ring hydrocarbon group that is classified as an alicyclic group, aromatic group, or heterocyclic group.

The term “alicyclic group” means a cyclic hydrocarbon group having properties resembling those of aliphatic groups.

The term “aromatic group” refers to mono-or polycyclic aromatic hydrocarbon groups. As used herein, an organic group can be substituted or unsubstituted.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring. This term also includes polypeptides that have been modified or derivatized, such as by glycosylation, acetylation, phosphorylation, and the like.

Herein, “isolated” as it refers to peptides (i.e., polypeptides) means that the peptides are derived from naturally occurring proteins or other polypeptides and free from other biological material or they are synthesized, either recombinantly or chemically.

The following abbreviations are used throughout the application:
A=Ala=Alanine T=Thr Threonine
V=Val=Valine C=Cys=Cysteine
L=Leu=Leucine Y=Tyr=Tyrosine
I=Ile=Isoleucine N=Asn=Asparagine
P=Pro=Proline Q=Gln=Glutamine
F=Phe=Phenylalanine D=Asp=Aspartic Acid
W=Trp=TryptophanE=Glu=Glutamic Acid
M=Met=Methionine K=Lys=Lysine
G=Gly=Glycine=Arg=Arginine
S=Ser=Serine H=His=Histidine

### BRIEF DESCRIPTION OF THE DRAWINGS

FIG. 1: Peptide sequence of human HLA class II molecule alpha chain, selectively used peptide domains were indicated as alpha 1, alpha 2 (a) and alpha 2 (b).

FIG. 2: HLA class II, alpha2 chain peptides (a) and (b) significantly reduced PBMC-proliferation after PHA-stimulation.

FIG. 3: IFNγ-production is significantly reduced by one HLA class II alpha 2 chain peptide (b), whereas alpha 2 (a) inhibits IFN gamma expression almost up to 50% in comparison to positive control.

FIG. 4: TIRC7 amino acid sequence (A) and Peptide sequences derived from TIRC7 molecule domains (B).

FIG. 5: TIRC7-peptides inhibit proliferation of PHA-stimulated PBMC.

FIG. 6: TIRC7-peptide P6 inhibits IFNγ-production significantly in coculture with PHA-stimulated PBMC.

FIG. 7: TIRC7-peptides do not induce any change in TNF alpha-production in coculture with PHA stimulated PBMC.

FIG. 8: TIRC7-peptides do not induce any change in IL4-production in coculture with PHA-stimulated PBMC.

### DETAILED DESCRIPTION OF ASPECTS AND PREFERRED EMBODIMENTS OF THE INVENTION

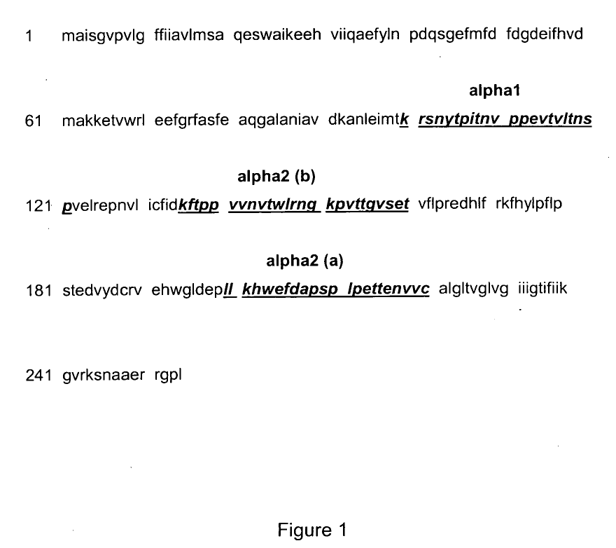

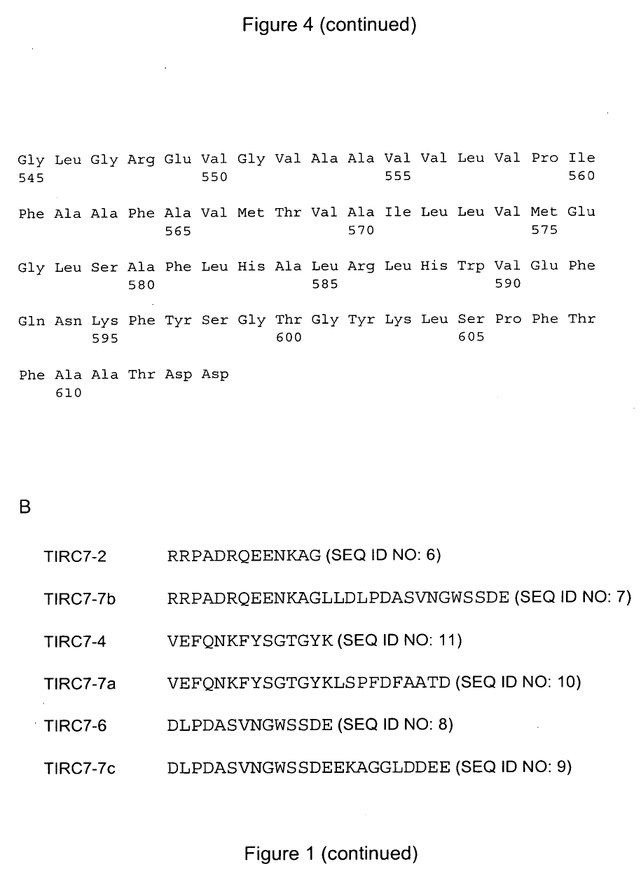

As mentioned before, in a first aspect the present invention provides HLA class II peptides capable of modulating the immune response. HLA-(Human Leukocyte associated Antigens) proteins play a major role in immuneresponse. It was shown earlier that different peptides derived from HLA-class II molecule alpha 1 chain region significantly regulated proliferation in several in vitro assays. Recent investigations by the inventor revealed a possible reaction between TIRC7 and HLA class II alpha chain as described in WO02/36149. In order to elucidate this binding and its consequences, three HLA class II-peptides were syntheticaly produced and examined regarding their functional activity on peripheral blood mononuclear cells (PBMCs) in vitro. Two of those peptides were derived from the HLA class II-alpha 2-chain (a and b) and one from the alpha1-chain (alpha 1) as shown in FIG. 1. In addition cytokine production was investigated to determine whether, like with TIRC7 antibodies, Th1-cytokine production would be inhibited by cocultivation with HLA II alpha chain 2 peptides.

Thus, the present invention provides isolated peptides derived from HLA class II alpha 2 chain that include a fragment of the amino acid sequence shown in FIG. 1 or analogs thereof that modulate the function of TIRC7 protein and/or at least one ligand thereof.

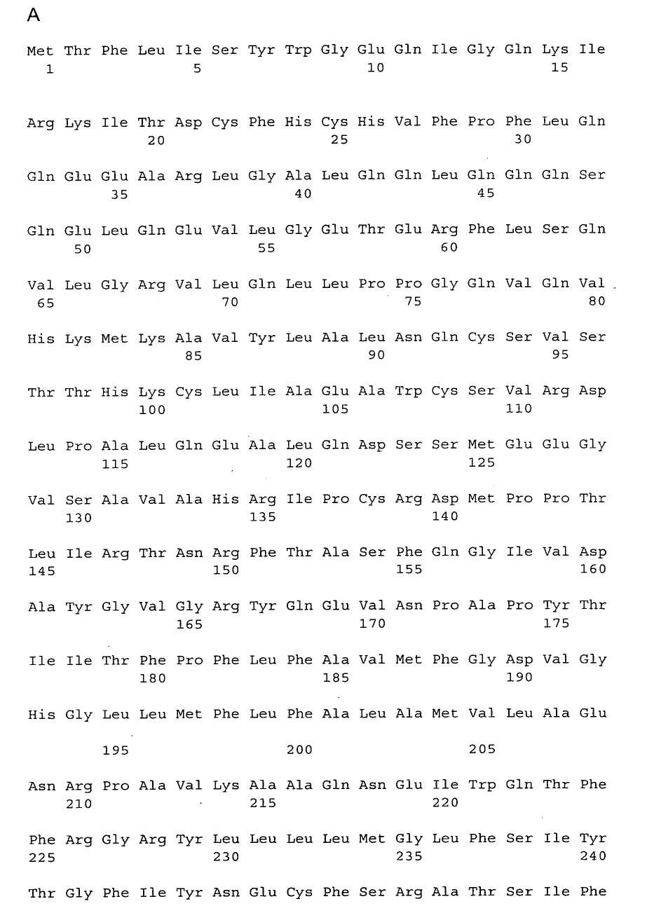

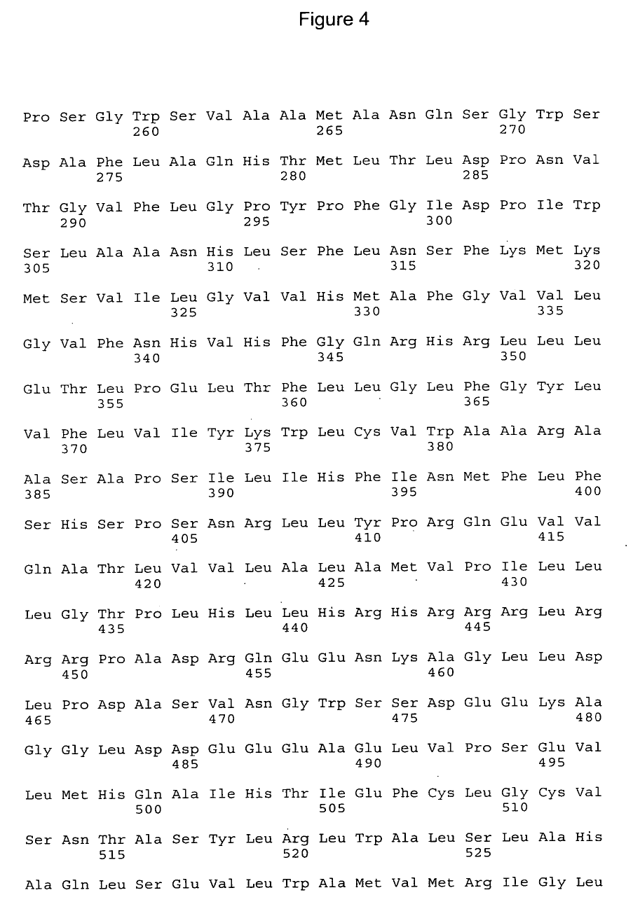

In a further aspect, the present invention provides TIRC7 peptides capable of modulating the immune response. As it was shown by Utku et al (Immunity, 1998) in vitro-translated protein TIRC7 signifincantly suppressed the proliferation of activated T-cells in MLR in a dose dependent manner. In order to investigate the effect of TIRC7-peptides on proliferation of stimulated PBMC similar assays were performed using 6 TIRC7-peptides of 13 to 29 amino acids (FIG. 4). In addition cytokine production was investigated to determine whether, like with the TIRC7 antibodies, Th1-cytokine production would be inhibited by cocultivation with TIRC7-peptides.

Thus, the present invention provides isolated peptides derived from TIRC7 that include an amino acid sequence represented by (at least one of) SEQ ID NOs: 6 to 10 or analogs thereof that modulate the function of TIRC7 protein and/or at least one ligand thereof.

Peptides were tested for their ability to inhibit proliferation of PHA-stimulated PBMCs. Two peptides alpha 2 (a) and 2(b) shown in FIG. 1 were found to be potent inhibitors of PBMC proliferation while another, alpha 1, had weaker if any activity. Furthermore, five TIRC7 peptides (SEQ ID NOs: 6 to 10) were found to be potent inhibitors of PBMC proliferation while another (SEQ ID NO: 11) had weaker if any activity. Accordingly, the peptides of the present invention are expected to be useful in the modulation of immune responses. Modulating the immune response, as for example by activating or inhibiting the proliferation and/or differentiation of T-cells, B-cells, NK cells, LAK cells, dendritic cells, monocytes, macrophages or other immune system cells, may be useful in treating autoimmune diseases, allergic diseases, and in transplantation therapies where graft vs. host or host vs. graft effects may be undesirable. The peptides could also be immune stimulants in settings such as cancer, infectious disease, sepsis, wound healing, or immunization. Alternatively, they could be immune suppressants. They could also be used to detect inflammation, and preferably modulate inflammation by activating or inhibiting activation of immune or inflammatory cells. A preferred method involves detecting (and preferably modulating) inflammation in tissues such as inflamed vasculature or leukocytes.

In one preferred embodiment the peptide and peptide conjugates are used for inducing or maintaining immune unresponsiveness in a subject. The term “immune unresponsiveness” comprises non-unresponsiveness of immune cell subsets like T-cell or B-cells, NK-cells, monocytes and/or macrophages.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Furthermore, the term “subject” as employed herein relates to animals in need of amelioration, treatment and/or prevention of immunological diseases as disclosed herein. Most preferably said subject is a human.

Thus, the present invention provides peptides derived from HLA class II alpha 2 chain and TIRC7 peptides that are capable of modulating (i.e., altering by increasing, decreasing, etc.), for example, immune cell activation, cell proliferation, cell differentiation, or binding of TIRC7 to its ligands. Preferably, the present invention provides isolated peptides comprising or consisting of an amino acid sequence indicated as alpha 2 (a) or alpha 2 (b) in FIG. 1, or a fragment thereof. In addition, the present invention provides isolated peptides represented by SEQ ID Nos: 6 to 10.

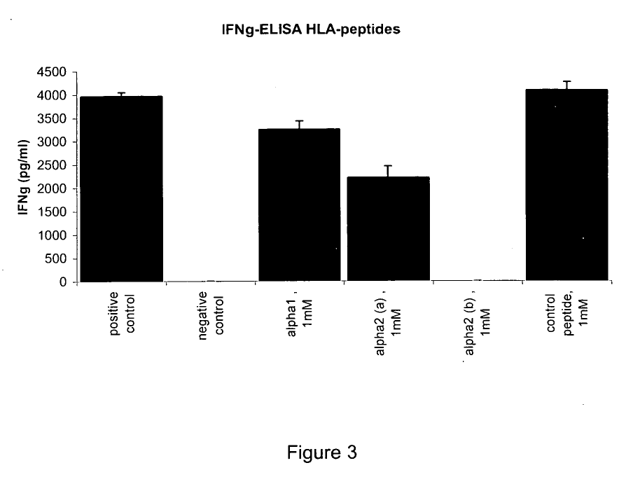

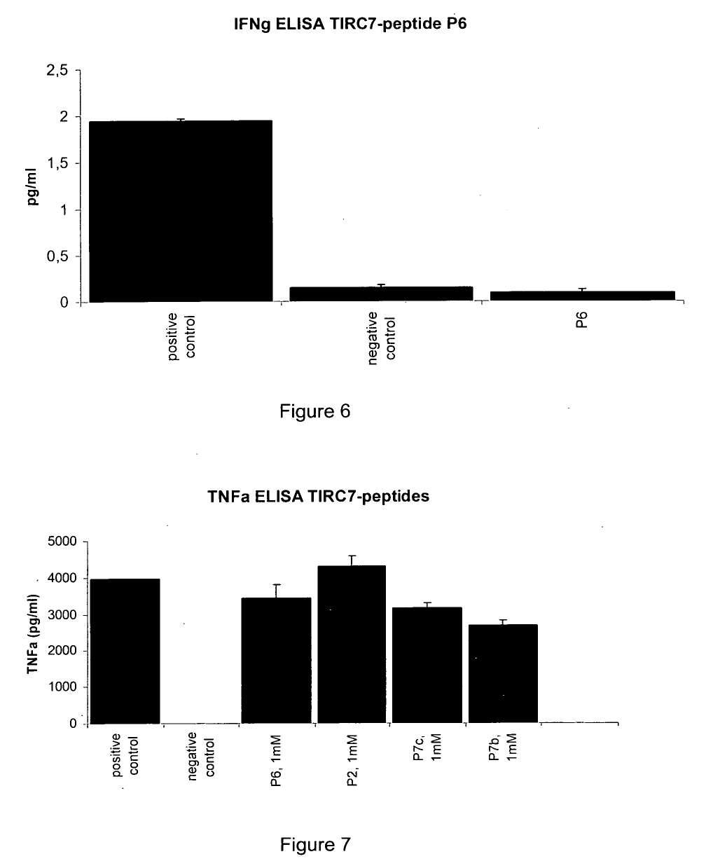

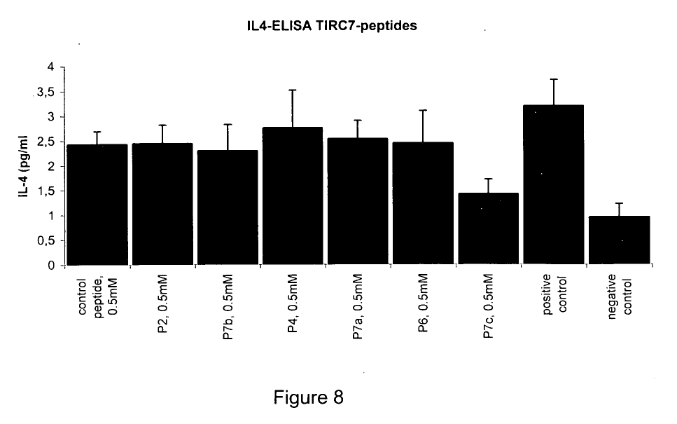

In addition, peptides of the present invention have been shown in PHA-stimulated PMBCs to inhibit IFNγ-production (see FIG. 3). Furthermore, TIRC7 peptides of the present invention have been shown in PHA-stimulated PMBCs (a) to inhibit IFNγ-production (see FIG. 6), (b) to not induce any change in TNF-α production (see FIG. 7), and (c) to not induce any change in IL4-production (see FIG. 8). Accordingly, the peptides of the invention are preferably used for specific inhibition or modulation of IFN-γ dependent immune reactions which refers to Th1 type of immune responses associated with transplant rejection, multiple sclerosis, Type I diabetes mellitus or rheumatoid arthritis without a modulatory effect on Th2 immune reactions. Others are believed to possess a least one activity as described herein.

Compositions comprising the polypeptides of this invention can be added to cells in culture (in vitro) or used to treat patients, such as mammals (in vivo). Where the polypeptides are used to treat a patient, the polypeptide is preferably combined in a pharmaceutical composition with a pharmaceutically acceptable carrier such as a larger molecule to promote polypeptide stability or a pharmaceutically acceptable buffer that serves as a carrier for the polypeptide or incorporated in a peptide conjugate that has more than one peptide coupled to a single entity.

The biological activity of the peptides identified here suggests that they have sufficient affinity to make them potential candidates for drug localization to cells expressing the appropriate surface structures. This targeting and binding to cells could be useful for the delivery of therapeutically active agents (including targeting drugs, DNA sequences, RNA sequences, lipids, proteins (e.g., human growth factors)) and gene therapy/gene delivery. More preferably, the therapeutically active agent is an, antiinflammatory agent.

Molecules/particles with a specific number of specific HLA II alpha 2 chain peptides or TIRC7 peptides would bind specifically to cells/tissues expressing specific ligand combinations, and therefore could have diagnostic and therapeutic use. Thus, the peptides of the present invention can be labeled (e.g., fluorescent, radioactive, enzyme, nuclear magnetic) and used to detect specific targets in vivo or in vitro including “immunochemistry” like assays in vitro. In vivo they could be used in a manner similar to nuclear medicine imaging techniques to detect tissues, cells, or other material expressing TIRC7 or its ligand.

The peptides of the present invention can be in their free acid form or they can be amidated at the C-terminal carboxylate group. The present invention also includes analogs of the polypeptide of, for example, those shown in FIG. 1, which typically have structural similarity with the corresponding amino acid sequences, preferably those of peptides alpha 2 (a) or alpha 2 (b). The present invention also includes analogs of TIRC7 peptides of, for example, SEQ ID NOs: 6 to 10, which typically have structural similarity with any one of SEQ ID NOs: 6 to 10. An “analog” of a polypeptide includes at least a portion of the polypeptide, wherein the portion contains deletions or additions of one or more contiguous or noncontiguous amino acids, or containing one or more amino acid substitutions. Substitutes for an amino acid in the polypeptides of the invention are preferably conservative substitutions, which are selected from other members of the class to which the amino acid belongs. An analog can also be a larger peptide that incorporates the peptides described herein. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For the purposes of this invention, conservative amino acid substitutions are defined to result from exchange of amino acids residues from within one of the following classes of residues: Class I: Ala, Gly, Ser, Thr, and Pro; Class II: Cys, Ser, Thr, and Tyr; Class III: Glu, Asp, Asn, and Gln (carboxyl group containing side chains): Class IV: His, Arg, and Lys (representing basic side chains); Class V: Ile, Val, Leu, Phe, and Met (representing hydrophobic side chains); and Class VI: Phe, Trp, Tyr, and His (representing aromatic side chains). The classes also include other related amino acids such as halogenated tyrosines in Class VI.

Polypeptide analogs, as that term is used herein, also include modified polypeptides. Modifications of polypeptides of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

In a preferred embodiment the peptide of the present invention comprises one of the group of D-isomer amino acids, L-isomer amino acids, or a combination thereof. The preparation of peptides comprising D-isomer amino acids is described for example in Schumacher, Science 271 (1996), 1854-1857.

A preferred polypeptide analog is characterized by having at least one of the biological activities described herein. Such an analog is referred to herein as a “biologically active analog” or simply “active analog”. The biological activity of a polypeptide can be determined, for example, as described in the Examples.

The polypeptides of the invention may be synthesized by the solid phase method using standard methods based on either t-butyloxycarbonyl (BOC) or 9 fluorenylmethoxy-carbonyl (FMOC) protecting groups. This methodology is described by G. B. Fields et al. in Synthetic Peptides: A User's Guide, W. M. Freeman & Company, New York, N.Y., pp. 77-183 (1992).

The present peptides may also be synthesized via recombinant techniques well known to those skilled in the art. For example, U.S. Pat. No. 5,595,887 describes methods of forming a variety of relatively small peptides through expression of a recombinant gene construct coding for a fusion protein which includes a binding protein and one or more copies of the desired target peptide. After expression, the fusion protein is isolated and cleaved using chemical and/or enzymatic methods to produce the desired target peptide.

The peptides of the present invention may be employed in a monovalent state (e.g., free peptide or peptide coupled to a carrier molecule or structure).

The peptides may also be employed as conjugates having more than one (same or different) peptide bound to a single carrier molecule. The carrier molecule or structure may be microbeads, liposomes, biological carrier molecule (e.g., a glycosaminoglycan, a proteoglycan, albumin, or the like), a synthetic polymer (e.g., a polyalkyleneglycol or a synthetic chromatography support), biomaterial (e.g., a material suitable for implantation into a mammal or for contact with biological fluids as in an extrcorporeal device), or other cell. Typically, ovalbumin, human serum albumin, other proteins, polyethylene glycol, or the like are employed as the carrier. Such modifications may increase the apparent affinity and/or change the stability of a peptide. The number of peptide fragments associated with or bound to each carrier can vary. In addition, as mentioned above, the use of various mixtures and densities of the peptides described herein may allow the production of complexes that have specific binding patterns in terms of preferred ligands. The polypeptides can be conjugated to other polypeptides using standard methods known to one of skill in the art. Conjugates can be separated from free peptide through the use of gel filtration column chromatography or other methods known in the art.

For instance, peptide conjugates may be prepared by treating a mixture of peptides and carrier molecules (or structures) with a coupling agent, such as a carbodiimide. The coupling agent may activate a carboxyl group on either the peptide or the carrier molecule (or structure) so that the carboxyl group can react with a nucleophile (e.g. an amino or hydroxyl group) on the other member of the peptide conjugate, resulting in the covalent linkage of the peptide and the carrier molecule (or structure).

As another example, peptides may be coupled to biotin-labeled polyethylene glycol and then coupled to avidin containing compounds. In the case of peptides coupled to other entities, it should be understood that the designed activity may depend on which end of the peptide is coupled to the entity.

The present invention also provides a composition that includes one or more active agents (i.e., peptides) of the invention and one or more pharmaceutically acceptable carriers. One or more polypeptides with demonstrated biological activity can be administered to a patient in an amount alone or together with other active agents and with a pharmaceutically acceptable buffer. The polypeptides can be combined with a variety of physiological acceptable carriers for delivery to a patient including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients.

The methods of the invention include administering to a patient, preferably a mammal, and more preferably a human, the composition of the invention in an amount effective to produce the desired effect. The peptides can be administered as a single dose or in multiple doses. Useful dosages of the active agents can be determined by comparing their in vitro activity and the in vivo activity in animal models. Methods for extrapolation of effective dosages in mice, and other animals, to humans are known in the art.

In a preferred embodiment, the pharmaceutical composition of the present invention comprises at least one second agent, preferably an agent which inhibits T-cell stimulation depending on the intended use. Such agents include, for example, molecules that are capable of blocking or mimicking receptor/ligand interaction or the like which leads to T-cell suppression.

Such agents comprise those blocking the activity of, e.g., costimulatory molecules, such as anti TIRC7 antibodies, and TNF-α blocking agents, e.g. antibodies, integrins, Ig-superfamily molecules, selectins as well as drugs blocking chemokines and their respective receptor interactions, drugs blocking IL2/IL2-receptor interaction and other conventional immunosuppressive drugs such as IL-2R mAbs, IL-Toxins and IL-Muteins. Examples for costimulatory molecules and their ligands are described in the prior art, e.g., in Schwartz, Cell 71 (1992), 1065-1068. The interruption of the receptor/ligand interactions by using mAbs or soluble CTLA41g for the interaction between CD28 to the B7-2 and CTLA4 to B7-1 and B7-2 are described in Blazar, J. Immunol. 157 (1996), 3250-3259; Bluestone, Immunity 2 (1995), 555-559; Linsley, Science 257 (1992), 792-95. Examples for blocking the receptor/ligand interaction by using mAbs to CD40 or CD40L are reported by Burden, Nature 381 (1996), 434-435; Kirk, Proc. Natl. Acad. Sci. USA 94 (1997), 8789-8794. CD2 antigen and its ligand LFA-3 are described in Bagogui Li et al., review in Adhesion Molecules, Fusion proteins, Novel Peptides, and Monoclonal Antibodies, Recent Developments in Transplantation Medicine, Vol. 11, 1995, Physicians&Scientists Publishing Co., Inc. and blocking of their interaction by using of mAbs (anti-Leu-5b, OKT11, T11) is reported in Brumberg, Transplantation 51 (1991) 219-225 or CD2.1gG1 fusion protein. The use of monoclonal Abs agains CD4 molecule is described in Cosimi, Surgery 108 (1990), 406-414. CD47 blockade by mAbs is described by Rheinhold, J. Exp. Med. 185 (1997), 1-11. Integrins and Ig-superfamily molecules include LFA-1 with its ligand ICAM-1, -2, -3, Mac-1 with ist ligand ICAM-1, -3; ICAM-1 with its ligand LFA-1, Mac-1, CD43; ICAM-2 with ist ligand LFA-1; ICAM-3 with its ligand LFA-1, Mac-1; VLA4 and VCAM-1 see, e.g., David, Adams, review in Adhesion Molecules, Fusion proteins, Novel Peptides, and Monoclonal Antibodies, Recent Developments in Transplantation Medicine, Vol. 11, 1995, Physicians&Scientists Publishing Co., Inc.; Isobe, Science, 255 (1992), 1125-1127; Cosimi, J. Immunology 144 (1990), 4604-4612; Hynes, Cell 69 (1992), 11-25.

Furthermore selectively interfering agents with VLA-4 mAbs to the alpha4 integrin chain (CD49d) can be used, beta1 integrin chain (CD29), or an activation-induced neo-epitope of VLA-4 as well as soluble VLA-4 ligands such as soluble fibronectin or its relevant peptide (GPEILDVPST), or soluble VCAM-1 or its relevant peptide. More selectively blocking agents are antisense oligonucleotides, designed to selectively hybridize with cytoplasmic alpha4, beta1, or VCAM-1 mRNA; Fedoseyeva, J. Immunol. 57 (1994), 606-612.

Another example is the drug pentoxifylline (PTX) that is able to block expression of VCAM-1; Besler, J. Leukoc. Biol. 40 (1986), 747-754. Furthermore, VCAM-1 mAb, M/K-2, anti-murine, for example could prolong allograft survival, Orosz, Transplantation, 56 (1993), 453-460.

Blocking of members of the integrin family and their ligands by using mAbs is decribed in Kupiec-Weglinski, review in Adhesion Molecules, Fusion proteins, Novel Peptides, and Monoclonal Antibodies, Recent Developments in Transplantation Medicine, Vol. 11, 1995, Physicians&Scientists Publishing Co., Inc.

Selectins, e.g., L-selectin (CD62L), E-selectin (CD62E), P-selectin (CD62P) have been described in Forrest and Paulson, Selectin family of adhesion molecules. In: Granger and Schmid-Schonbein, eds. Physiology and Pathophysiology of Leukocyte Adhesion. New York, Oxford Press, 1995, pp 68-146.

The combination of conventional immunosuppressive drugs, e.g., ATG, ALG, OKT3, Azathioprine, Mycophenylate, Mofetyl, Cyclosporin A, FK506, Sirolimus (Rapamune), Corticosteroids may be used as decribed in Cosimi, Transplantation 32 (1981), 535-539; Shield, Transplantation 38 (1984), 695-701, and Graft, June 2001, Vol 4 (4).

The interruption of chemokines and interactions with their respective receptor by using mAbs is reviewed in Luster, Chemokines-chemotactic cytokines that mediate inflammation, New Engl. J. Med. Feb. (1998), 436-445.

Thus, any agent as defined above and referenced by way of example can be used in accordance with the pharmaceutical composition of the invention or the methods and uses described herein.

The agents of the present invention are preferably formulated in pharmaceutical compositions and then, in accordance with the methods of the invention, administered to a patient, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, intraorgan, intraarterial and intravenous) administration.

Formulations suitable for parenteral administration conveniently include a sterile aqueous preparation of the active agent, or dispersions of sterile powders of the active agent, which are preferably isotonic with the blood of the recipient. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder or granules, as liposomes containing the active agent, or as a solution or suspension in an aqueous liquor or non-aqueous liquid such as a syrup, an elixir, an emulsion, or a draught. Such compositions and preparations typically contain at least about 0.1 wt-% of the active agent. The amount of polypeptide (i. e., active agent) is such that the dosage level will be effective to produce the desired result in the patient.

Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

Furthermore, the present invention relates to the use of a peptide comprising an amino acid sequence from TIRC7 protein for the preparation of a pharmaceutical composition for inhibition of an immune response, preferably for the treatment of graft versus host disease, autoimmune diseases, allergic diseases, infectious diseases, sepsis, for the treatment of tumors, for the improvement of wound healing or for inducing or maintaining immune unresponsiveness in a subject.

The examples illustrate the invention.

### EXAMPLES

### Example 1

### Cell Proliferation ELISA

PBMC of healthy donors were isolated according to the Ficoll-Paque density centrifugation protocol. Samples of 50000 PBMC's/well were stimulated with PHA (1 μg/ml) and incubated for 48 h at 5% CO₂, 37° C. in presence of HLA-peptides and controls (BSA) in a total volume of 100 μl/well. Samples were run in triplicates on 96 well-microtiter-plates (MTP's). After 48 h BrdU-labeling solution provided with the Cell Proliferation ELISA Kit, Roche, was added and cells were reincubated for additional 18 h. MTP's were then centrifuged at 300×g for 10 min and labeling medium was removed by flicking off. After removal of the labeling medium the cells were dried at 60° C. for 1 h. For cell fixation and DNA denaturation FixDenat solution was added and incubated for 30 min at room temperature. FixDenat solution was removed thouroughly by flicking off and tapping. Anti-BrdU-Peroxidase conjugate was added and incubated for 1.5 h at room temperature. Excessive conjugate was removed by washing 3 times with washing buffer. Afterwards substrate solution was added and incubated until colour development was sufficient for photometric detection at 370 nm.

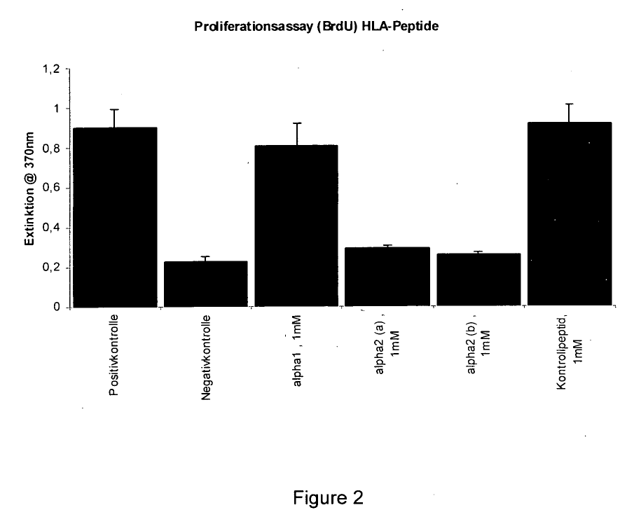

As shown in FIG. 2 HLA2-peptides derived from the alpha2-chain significantly inhibited proliferation of PHA-stimulated PBMC in vitro. In contrast there was no effect observed with the alpha 1-chain peptide.

### Example 2

### Quantitation of Secreted Cytokines in the Supernatant

PBMC of healthy donors were isolated according to the Ficoll-Paque density centrifugation protocol. Samples of 50000 PBMC's/well were stimulated with PHA (1 g/ml) and incubated for 48 h at 5% CO₂, 37° C. in presence of HLA-peptides and controls (BSA) in a total volume of 100 μl/well. Samples were run in triplicates on 96 well-microtiter-plates (MTP's).

After 48 h MTP's were centrifuged at 300×g for 10 min and supernatants collected from the wells. The quantitation of Interferon-γ (IFNg) in the supernatant was carried out on anti-IFNg-antibody-coated microtiter strips provided with the Cytoscreen® ELISA Kit, Biosource. The formerly collected supernatants (diluted 1:2 in dilution buffer) and diluted standards were incubated in presence of a biotinylated secondary antibody recognizing IFNg for 1.5 h at room temperature on these strips. Afterwards excessive secondary antibody was removed by washing 3 times with washing buffer. A streptavidin-peroxidase conjugate was added and incubated for 45 min at room temperature. Excessive conjugate was removed by washing. TMB-substrate-solution was added and the strips incubated for additional 30 min in the dark followed by the addition of stop solution. The colour development was measured at 450 nm and the numbers were statistically analyzed.

As shown in FIG. 3 HLA2-alpha2-chain-peptide (b) significantly inhibited IFNg-poduction whereas an reduction up to 50% was observed with alpha2-chain-peptide (a) of PHA-stimulated PBMC in vitro. In contrast there was no effect observed with the alpha1-chain peptide.

Interestingly the alpha2-chain of HLA2 was identified to interact with TIRC7 as possible ligand in different binding studies as demonstrated in WO02/36149 by the inventor. Based on these prior shown experiments and the results presented in accordance with the present invention, one might hypothesize that the Th 1-like cytokine IFNγ-production and the proliferation inhibiting effect of HLA class II alpha 2 chain derived-peptides are mediated through the signalling of TIRC7.

### Example 3

### Radioactive Proliferation Assay-Incorporation of ³H-thymidine

PBMC of healthy donors were isolated according to the Ficoll-Paque density centrifugation protocol. Samples of 50000 PBMC's/well were stimulated with PHA (1 μg/ml) and incubated for 48 h at 5% CO₂, 37° C. in presence of TIRC7-peptides (see FIG. 4(B)) and controls (BSA) in a total volume of 10011/well. Samples were run in triplicates on 96 well-microtiter-plates (MTP's). After 48 h 0.5 μCi ³H-thymidine per well were added and the cells were reincubated for additional 18 h. Cells were harvested and lysed using a cell harvester and collected on nitrocellulose-filter-MTP's. Plates were dried at room temperature for 3 h. To enhance the radioactive signal produced by the samples a scintillation fluid was added and counts per minute were measured with a beta counter.

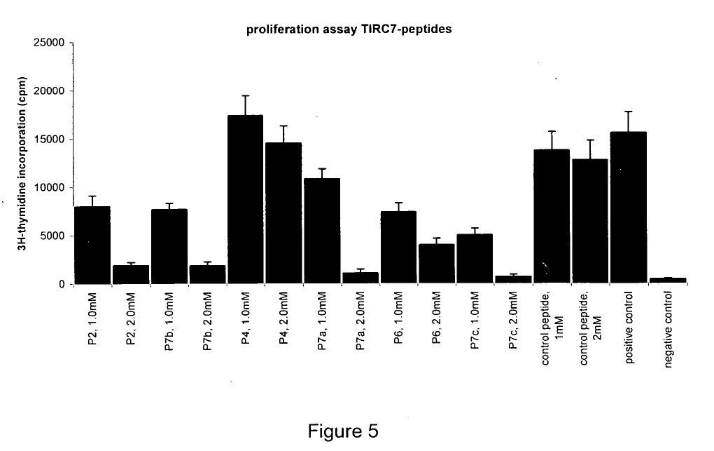

As shown in FIG. 5, except peptide P4 all TIRC7-peptides inhibited proliferation of PHA-stimulated PBMC significantly in a dose dependent manner whereas the control peptide showed no effect on proliferation.

### Example 4

### Quantitation of Secreted Cytokines in PBMC-Supernatants

PBMC of healthy donors were isolated according to the Ficoll-Paque density centrifugation protocol. Samples of 50000 PBMC's/well were stimulated with PHA (1 μg/ml) and incubated for 48 h at 5% CO₂, 37° C. in presence of TIRC7-peptides and controls (BSA) in a total volume of 100 μl/well. Samples were run in triplicates on 96 well-microtiter-plates (MTP's). After 48 h MTP's were centrifuged at 300×g for 10 min and supernatants collected from the wells. The quantitation of cytokines in the supernatant was carried out on anti-cytokine-antibody-coated microtiter strips provided with the Cytoscreen® ELISA Kit, Biosource. The formerly collected supernatants and diluted standards were incubated in presence of a biotinylated secondary antibody recognizing the specific cytokine for 1,5-3 h at room temperature on these strips depending on the detemined cytokine. Afterwards excessive secondary antibody was removed by washing 3 times with washing buffer. A streptavidin-peroxidase conjugate was added and incubated for 45 min −1 h at room temperature. Excessive conjugate was removed by washing. TMB-substrate-solution was added and the strips incubated for additional 30 min in the dark followed by the addition of stop solution. The colour development was measured at 450 nm and the numbers were statistically analyzed.

As shown in FIG. 6, quantification of cytokines in coculture supernatants of TIRC7-Peptides with PHA-stimulated PBMC revealed that IFNγ-production was significantly inhibited by TIRC7-peptides, shown as an example for P6. No changes in cytokine production were observed with TNFα and IL4 in comparison with appropriate controls; see FIGS. 7 and 8.

The complete disclosure of all patents, patent documents, and publications cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A peptide capable of inhibiting proliferation of peripheral blood mononuclear cells (PBMCs), comprising a fragment of the amino acid sequence from HLA-(Human Leukocyte associated Antigen) class II alpha 2 chain.

2. The peptide of claim 1, which comprises a fragment of the amino acid sequence shown in SEQ ID NO: 1.

3. The peptide of claim 1 comprising or consisting of an amino acid sequence shown in SEQ ID NO: 3 or SEQ ID NO: 4, or a fragment thereof.

4. A peptide capable of inhibiting proliferation of peripheral blood mononuclear cells (PBMCs), comprising a fragment of the amino acid sequence from T cell immune response cDNA 7 (TIRC7) protein.

5. The peptide of claim 4, which comprises a fragment of the amino acid sequence shown in SEQ ID NO: 5.

6. The peptide of claim 4 comprising or consisting of an amino acid sequence as shown in any one of SEQ ID NOs: 6 to 10, or a fragment thereof.

7. The peptide of claim 1, wherein said peptide comprises one of the group of D-isomer amino acids, L-isomer amino acids, or a combination thereof.

8. A composition comprising at least one peptide of claim 1.

9. The composition of claim 8 which is a pharmaceutical composition and further comprises a pharmaceutically acceptable carrier.

10. The pharmaceutical composition of claim 9 further comprising an immunosuppressive agent.

11. Use of a peptide comprising a fragment of the amino acid sequence from HLA class II alpha 2 chain or a fragment of the amino acid sequence of TIRC7 for the preparation of a pharmaceutical composition for inhibition of an immune response.

12. The use of claim 11, wherein said pharmaceutical composition is administered intravenous, intramuscular, subcutaneous, intraperitoneal, or as an aerosol.

13. A method of modulating the immune response in a subject in need thereof, comprising administering a peptide of claim 1.
